(19) United States
(12) Patent Application Publication
(10) Pub. No.: US 2024/0398619 A1
(43) Pub. Date: Dec. 5, 2024

(54) Surgical Apparatus

(71) Applicant: Nallakrishnan Family Trust, Willowbrook, IL (US)

(72) Inventor: Ravi Nallakrishnan, Willowbrook, IL (US)

(21) Appl. No.: 18/688,155

(22) PCT Filed: Sep. 13, 2022
(86) PCT No.: PCT/US2022/043286

§ 371 Date: Feb. 29, 2024

Related U.S. Application Data
(60) Provisional application No. 63/249,661, filed on Sep. 29, 2021.

(51) Int. Cl. A61F 9/007

(52) U.S. Cl. CPC A61F 9/00745; A61B 2217/005; A61B 2217/007

(57) ABSTRACT

A hollow tip (10, 10A, 10B, 10C, 10D, 10E, 10F, 10G) for use with a cannula (20, 20D, 20E, 20F, 20G) includes a sheath portion (30, 30A, 30B, 30C, 30D, 30E, 30F, 30G) for being affixed around an end of the cannula (20, 20D, 20E, 20F, 20G) and defining a central axis (38, 38A, 38B, 38C, 38D, 38E, 38F, 38G). The tip (10, 10A, 10B, 10C, 10D, 10E, 10F, 10G) includes a terminal portion (34, 34A, 34B, 34C, 34D, 34E, 34F, 34G) extending from the sheath portion (30, 30A, 30B, 30C, 30D, 30E, 30F, 30G) along the central axis (38, 38A, 38B, 38C, 38D, 38E, 38F, 38G). The terminal portion (34, 34A, 34B, 34C, 34D, 34E, 34F, 34G) and the sheath portion (30, 30A, 30B, 30C, 30D, 30E, 30F, 30G) define an internal passage (42, 42A, 42B, 42D, 42E, 42F, 42G) communicating with at least one port (46, 46A, 46B, 46C, 46D, 46E, 46F, 46G) in the terminal portion (34, 34A, 34B, 34C, 34D, 34E, 34F, 34G) to permit fluid communication through the tip (10, 10A, 10B, 10C, 10D, 10E, 10F, 10G). The terminal portion (34, 34A, 34B, 34C, 34D, 34E, 34F, 34G) includes at least one recess (54, 54A, 54B, 54C) therein for indicating the location of the at least one port (46, 46A, 46B, 46C, 46D, 46E, 46F, 46G) and/or inhibiting adherence of eye tissue to the tip (10, 10A, 10B, 10C, 10D, 10E, 10F, 10G).

### PRIORITY

This application claims priority of U.S. Provisional Patent Application No. 63/249,661, filed Sep. 29, 2021, the entire contents of which is incorporated herein by reference.

### FIELD OF THE INVENTION

The present invention relates generally to surgical instruments used in ophthalmic surgery and, more particularly, to improved irrigation and aspiration instruments.

### BACKGROUND OF THE INVENTION

Phacoemulsification has come to be a technique of choice for the removal of damaged or diseased lenses from the eye. Commonly, such surgery is called for when a patient develops cataracts, a condition in which a portion of the eye lens becomes hard and opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. The lens of the eye is held within a capsular bag positioned behind the iris in the anterior chamber of the eye. When the lens becomes damaged or diseased, a common surgical technique is to remove the lens and replace it with an artificial intraocular lens (IOL). Removal of the lens is commonly carried out by physical dismemberment of the lens and phacoemulsification, that is, using an instrument to which vibrational energy is transmitted by a phacoemulsification hand piece. The natural lens may also be removed by way of femtosecond laser which softens or dismembers the lens.

During phacoemulsification, the lens is broken into fragments and the fragments are emulsified and then removed from the capsular bag by aspirating the fragments through a cannula formed as part of the phacoemulsification instrument. After the lens has been removed, the capsular bag must be cleaned in order to prepare it for the insertion of an IOL. In particular, epithelial, and cortical tissue fragments must be removed from the floor and perimeter of the capsular bag. Typically, an irrigation and aspiration tip is coupled to a hand piece, which may be the phacoemulsification hand piece, for removal of the fragments. The hand piece will typically have an irrigation and aspiration mode specifically for this task. U.S. patent application Ser. No. 15/190,366, hereby incorporated by reference in its entirety, discloses an irrigation and aspiration apparatus have any enlarged, ball-like tip with one or more irrigation or aspiration ports, and a sheath portion that locks into a recess on the outer surface of the cannula.

The inventor has found that users may have a difficult time identifying or locating the irrigation or aspiration port in the tip. Furthermore, the inventor has found that some prior art tips have been prone to sticking of the emulsified or laser dismembered nucleus tissues, which may inhibit efficient aspiration and decrease the efficiency of the operation.

Complete removal of the cortex is important for several reasons. If the cortex is not completely removed it may cause post-operative inflammation and an increase in intraocular pressure. Incomplete removal of the cortex may also cause decentration or tilting of the IOL which, in turn, would cause a postoperative refractive error or induced astigmatism. This is especially important if the IOL is a multi-focal or toric type. Incomplete removal of the cortex may also result in the formation of after (secondary) cataract which would impair vision and require laser capsulotomy.

The present invention is directed to an irrigation and aspiration apparatus, tip, tip and cannula combination, or assembly with a handpiece which has been specifically configured to promote safe and efficient irrigation and aspiration of the capsular bag attendant to eye surgery such as implantation of an intraocular lens and to provide improved visibility of the port location or locations in the tip and/or to prevent or inhibit sticking or adhesion of the nucleus to the tip.

### SUMMARY OF THE INVENTION

The present invention is directed to an improved surgical apparatus which is particularly suited for use with an associated irrigation and/or aspiration handpiece.

In accordance with one broad form of the present invention, the apparatus has the form of a hollow tip that is provided for use with a cannula to facilitate an irrigation and/or aspiration surgical operation in the lens capsule of the eye. The tip includes a sheath portion for being affixed around an end of the cannula. The sheath portion defines a central axis. The tip from which a terminal portion extends. The terminal portion and the sheath portion define an internal passage communicating with at least one port in the terminal portion to permit fluid communication through the tip. The terminal portion includes at least one recess therein for indicating the location of the at least one port and/or inhibiting adherence of eye tissue to the tip.

In broad form of the present invention, the tip is in combination with a cannula. More preferably, the tip and cannula are assembled or otherwise attached to a handpiece connected to a vacuum source and/or an infusion fluid supply source.

In one preferred form of the present invention, the least one recess has the form of a plurality of recesses on the terminal portion of the tip. Preferably, the plurality of recesses extend along an arc extending through the geometric center of the at least one port on the outer surface of the terminal portion.

In yet another preferred form of the present invention, the plurality of recesses are circular or semi-spherical.

In yet another preferred form of the present invention, the plurality of recesses are elongate or linear in shape.

In still another preferred form of the present invention, the plurality of recesses cover substantially all, or a majority, of the terminal portion outer surface.

In yet another preferred form of the present invention, the sheath portion defines a first diameter, and the terminal portion defines a second diameter which is greater (longer) than the first diameter.

According to one preferred form of the present invention, the at least one port is angled between about 40 degrees and about 50 degrees relative to the central axis, more preferably about 45 degrees.

According to one preferred form of the present invention, the tip is formed from an elastomer that is substantially less rigid than the material of the associated cannula (which can be metal or a rigid thermoplastic). In some forms, the tip is formed from a rigid thermoplastic.

In another form of the present invention, the terminal portion of the tip has a spherical configuration.

In another form of the present invention, the terminal portion of the tip has an ovoid configuration.

In yet another form of the present invention, the terminal portion of the tip has a semi-ovoid configuration.

In one form of the present invention, the terminal portion of the tip has a truncated, flat end.

In another preferred form of the present invention, the terminal portion defines an outer surface that is relatively rougher than the sheath portion.

### BRIEF DESCRIPTION OF THE DRAWINGS

In the accompanying drawings forming part of the specification, in which like numerals are employed to designate like parts throughout the same,

FIG. 1 is a greatly enlarged, elevational view of a first illustrated embodiment of an irrigation and/or aspiration tip of the present invention for being assembled at the distal end of a cannula of a surgical apparatus for being attached to a handpiece that is connected to a vacuum source and, optionally, is connected to an irrigation fluid supply source;

FIG. 2 is a greatly enlarged, isometric view of the tip of FIG. 1 assembled at the distal end of a fragmentary portion of a cannula;

FIG. 3 is a greatly enlarged, isometric view of the tip of FIG. 1;

FIG. 4 is a greatly enlarged, top plan view of the tip of FIG. 1;

FIG. 5 is a greatly enlarged, elevational view of the tip of FIG. 1, and FIG. 5 shows the interior structure of the tip in hidden lines;

FIG. 6 is a greatly enlarged, elevational view of the tip of FIG. 1, and FIG. 5 shows the interior structure of the tip in hidden lines;

FIG. 7 is a greatly enlarged, front elevational view of the tip of FIG. 1;

FIG. 8 is a greatly enlarged, bottom plan view of the tip of FIG. 1;

FIG. 9 is a greatly enlarged, right-side elevational view of the tip of FIG. 1;

FIG. 10 is a greatly enlarged, cross-sectional view of the tip of FIG. 1, taken along the along a vertical plane 10-10 in FIG. 9 extending through a central axis of the tip;

FIG. 11 is a greatly enlarged, elevational view of a second illustrated embodiment of an irrigation and/or aspiration tip of the present invention for being assembled at the distal end of a cannula of a surgical apparatus for being attached to a handpiece that is connected to a vacuum source and, optionally, is connected to an irrigation fluid supply source;

FIG. 12 is a greatly enlarged, front elevation view of the tip of FIG. 11;

FIG. 13 a greatly enlarged, top plan view of the tip of FIG. 11;

FIG. 14 is a greatly enlarged, elevational view of the tip of FIG. 11, and FIG. 14 shows the interior structure of the tip in hidden lines;

FIG. 15 is a greatly enlarged, elevational view of the tip of FIG. 11;

FIG. 16 is a greatly enlarged, front elevational view of the tip of FIG. 11;

FIG. 17 is a greatly enlarged, bottom plan view of the tip of FIG. 11;

FIG. 18 is a greatly enlarged, right-side elevational view of the tip of FIG. 11;

FIG. 19 is a greatly enlarged, cross-sectional view of the tip of FIG. 11, taken along the along a vertical plane 19-19 in FIG. 18 extending through a central axis of the tip;

FIG. 20 is a greatly enlarged, front elevational view of a third illustrated embodiment of an irrigation and/or aspiration tip of the present invention for being assembled at the distal end of a cannula of a surgical apparatus for being attached to a handpiece that is connected to a vacuum source and, optionally, is connected to an irrigation fluid supply source;

FIG. 21 is a greatly enlarged, elevational view of the tip of FIG. 20;

FIG. 22 a greatly enlarged, top plan view of the tip of FIG. 20;

FIG. 23 is a greatly enlarged, elevational view of the tip of FIG. 20, and FIG. 23 shows the interior structure of the tip in hidden lines;

FIG. 24 is a greatly enlarged, elevational view of the tip of FIG. 20;

FIG. 25 is a greatly enlarged, front elevational view of the tip of FIG. 20;

FIG. 26 is a greatly enlarged, bottom plan view of the tip of FIG. 20;

FIG. 27 is a greatly enlarged, right-side elevational view of the tip of FIG. 20;

FIG. 28 is a greatly enlarged, cross-sectional view of the tip of FIG. 20, taken along the along a vertical plane 28-28 in FIG. 27 extending through a central axis of the tip;

FIG. 29 is a greatly enlarged, isometric view, taken from the top and rear, of a fourth illustrated embodiment of an irrigation and/or aspiration tip of the present invention for being assembled at the distal end of a cannula of a surgical apparatus for being attached to a handpiece that is connected to a vacuum source and, optionally, is connected to an irrigation fluid supply source;

FIG. 30 is a greatly enlarged, cross-sectional view of a fifth illustrated embodiment of an irrigation and/or aspiration tip of the present invention, taken through a vertical plane extending through the central axis of the tip and the aspiration port;

FIG. 31 is a greatly enlarged, cross-sectional view of a sixth illustrated embodiment of an irrigation and/or aspiration tip of the present invention, taken through a vertical plane extending through the central axis of the tip and the aspiration port;

FIG. 32 is a greatly enlarged, cross-sectional view of a seventh illustrated embodiment of an irrigation and/or aspiration tip of the present invention, taken through a vertical plane extending through the central axis of the tip and the aspiration port; and

FIG. 33 is a greatly enlarged, cross-sectional view of an eighth illustrated embodiment of an irrigation and/or aspiration tip of the present invention, taken through a vertical plane extending through the central axis of the tip and the aspiration port.

### DETAILED DESCRIPTION OF THE PREFERRED EMBODIMENTS

While the present invention is susceptible of embodiment in various forms, there are shown in the drawings and will hereinafter be described the presently preferred embodiments, with the understanding that the present disclosure should be considered as an exemplification of the invention, and is not intended to limit the broadest forms of the invention to only the specific embodiments illustrated.

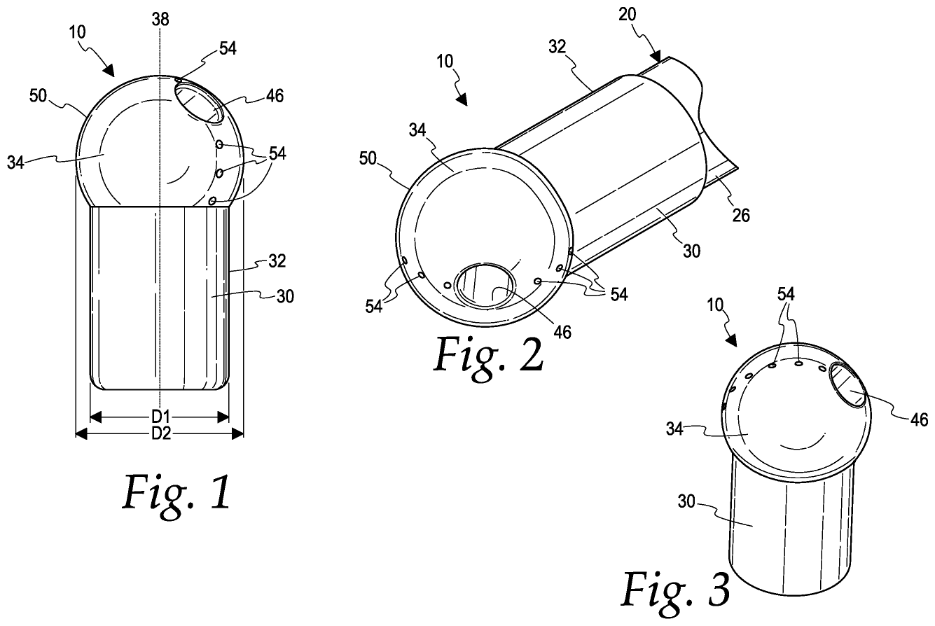

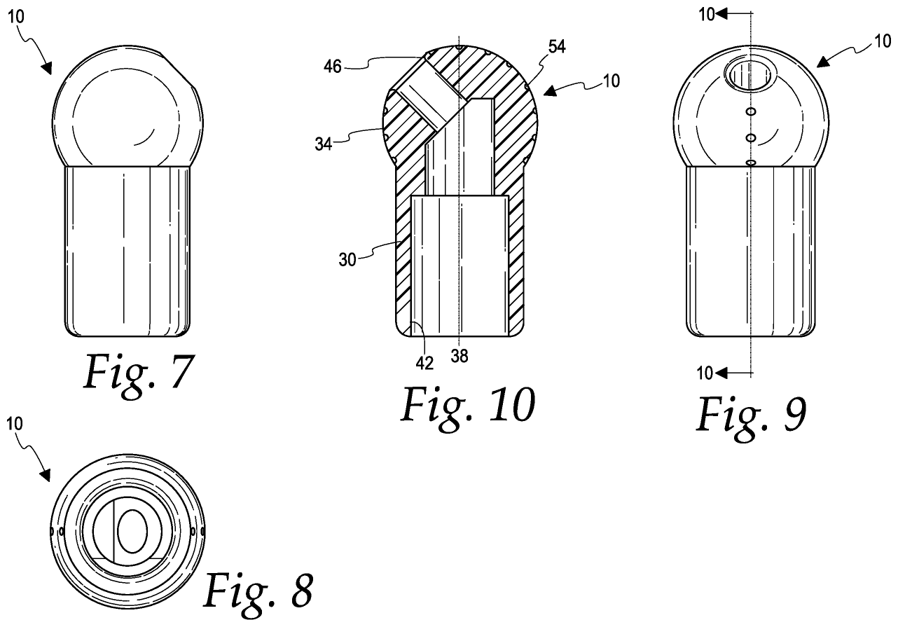

A first illustrated embodiment of a surgical apparatus or irrigation/aspiration tip 10 according to the present invention is shown in FIGS. 1-10, wherein the tip 10 is configured for being assembled with a hollow cannula or shaft 20 (FIG. 2 only) for being connected to an irrigation handpiece or irrigation/aspiration handpiece. The cannula 20 defines an internal passage through which aspiration or irrigation is effected during surgery when the cannula 20 is connected to a vacuum source or a fluid supply source via the handpiece. It will be understood that the cannula 20 may have any shape or structure (straight, angled, etc.) and may have a variety of means or structures suited for being attached to a variety of handpieces, such as by mating screw threads, clamps, locks, friction fitting, etc. Suitable structures of instruments are described in U.S. Pat. No. 8,764,782, the entirety of which is incorporated herein by reference. The cannula 20 is preferably formed from a sufficiently rigid metal or hard thermoplastic material. Other materials may be used.

Referring now to FIG. 2, the tip 10 further is hollow for being attached around the distal end 26 of the cannula 20. Importantly, the tip 10 is formed from a polymer, preferably an elastomer such as silicone, which is substantially softer and less rigid than the material of the cannula 20 (which may be metal or a rigid or hard polymer) to permit the tip 10 to safely contact the lens capsule during use.

With reference FIGS. 2 and 6, the tip 10 includes a cylindrical sheath portion 30 for receiving the open distal end 26 (FIG. 2) of the cannula 20 (FIG. 2) and a rounded end or terminal portion 34 that extends distally outwardly of the sheath portion 30 along a central axis 38 defined by the sheath portion 30. The sheath portion 30 includes a cylindrical outer surface 32 which defines a first diameter D1 in radial direction relative to the central axis 38. Each of the sheath portion 30 and the terminal portion 34 is hollow, defining an internal passage 42 (FIG. 6) therein communicating with a port 46 which fluidly connects to the passage of the cannula 20 to facilitate aspiration of fluids and tissues through the tip 10 when subjected to a vacuum or flow of irrigation fluid when connected to a fluid supply source. The port 46 is circular, and is angled about 45 degrees relative to the central axis 38 of the sheath portion 30 (as can be seen in FIG. 10). It will be understood that the port 46 may be located at any angle along a 360 degree sector on the terminal portion 34. For example, the port 46 may be aligned with the central axis 38 or angled 20 degrees or 120 degrees relative to the central axis 38. In other forms, the port 46 may have cross-sectional shapes other than circular, such as polygonal, oval, conical or tapering up or tapering down, etc. Furthermore, it will be understood that the terminal portion 34 may be provided with more than one port 46, and such plurality of ports 46 may be angled in a symmetric or asymmetric configuration relative to the central axis 38.

Referring next to FIG. 1, the terminal portion 34 defines an enlarged spherical outer surface 50 that defines a second diameter D2 in radial direction relative to the central axis 38, which is substantially greater (more than 10%) in length than the length of the first diameter D1 of the sheath.

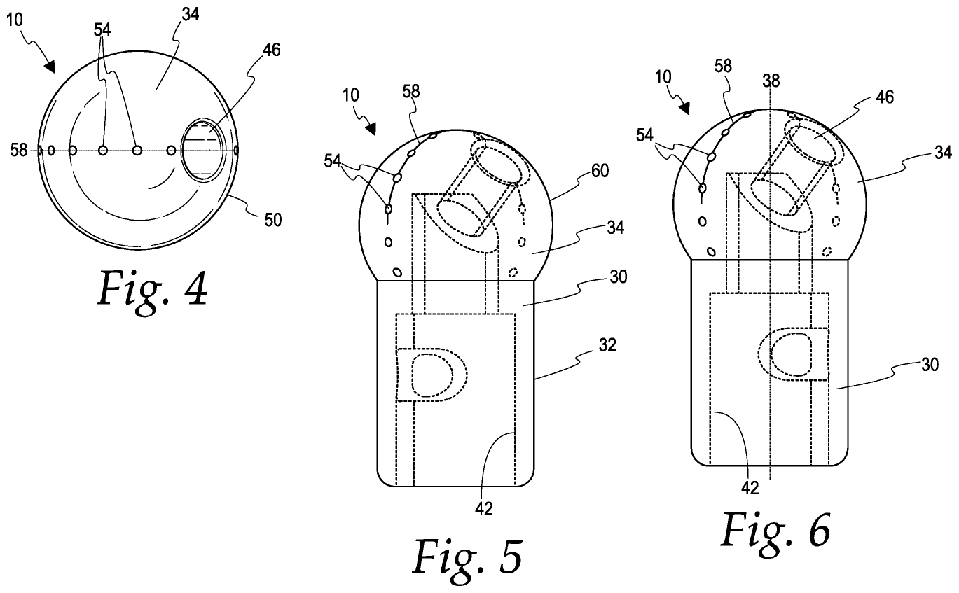

Referring next to FIG. 4, the outer surface 50 of the terminal portion 34 defines a plurality of circular recesses 54 therein which are arranged in an arc 58 that extends through the center of the port 46. In one preferred form of the invention, the port 46 has a nominal diameter of about 0.30 mm and the recesses 54 each have a diameter of about 0.05 mm.

A desirable feature of the present instrument tip 10 is that the circular recesses 54 serve to indicate to a user the location of the port 46 within the terminal portion 34, which may be obscured or not visible during use. Furthermore, the circular recesses 54 in the terminal portion 34 function to prevent or at least minimize the adhesion of the emulsified nucleus during use of the tip 10.

It is contemplated that the outer surface 50 of the tip 10 may have a smooth or a roughened surface for polishing of the lens capsule.

The tip 10 and cannula 20 may be connected to a handpiece and an irrigation fluid supply (with or without a sleeve) and/or a vacuum supply. Accordingly, the tip may function solely as an aspiration device, solely as an irrigation device, or a function as an aspiration and irrigation device when coupled with a sleeve. The sleeve may have any form. One such form is the elastomeric sleeve which is coaxially fitted around the cannula 20 to supply fluid proximate to the distal end 26 thereof.

The inventor has found that the inventive tip is especially beneficial in aspiration of the cortex while simultaneously keeping the iris and the posterior capsule away from the aspiration port. Furthermore, the inventor has found that the inventive tip minimizes chamber flutter during operation even with a relatively high irrigation fluid flow rate and a relatively high vacuum, even with a small pupil. It is believed that the shape of the tip advantageous for polishing of the posterior capsule in a safe and efficient manner. The tip 10 further provides the user with a better visibility and control of the location of the port 46 compared to prior art devices. In some forms, the recesses 54 may instead have the form of pigmentation, dye, or other marking on the surface 50 of the tip 10.

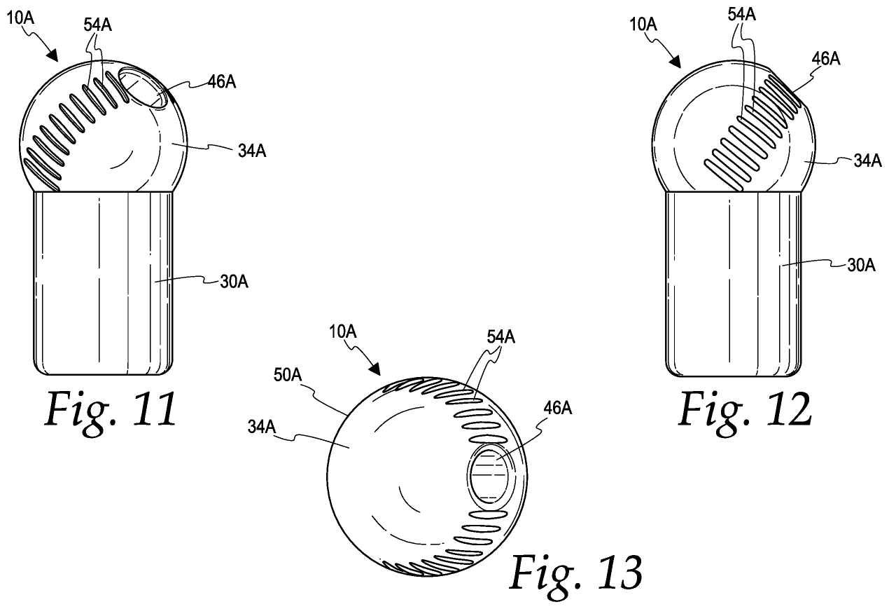

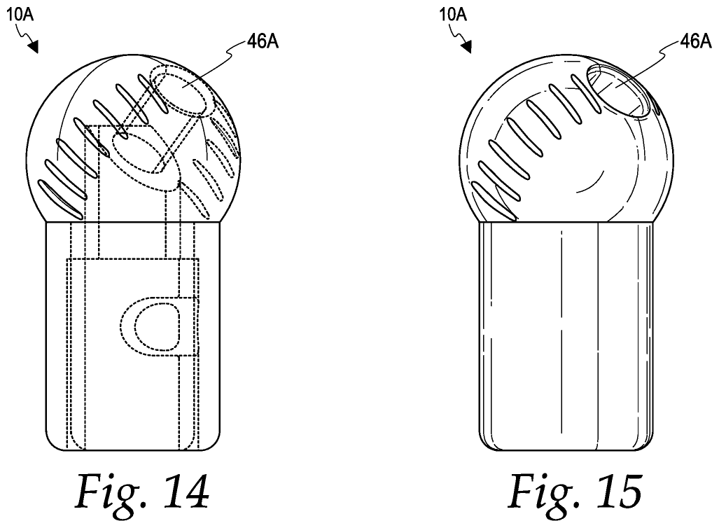

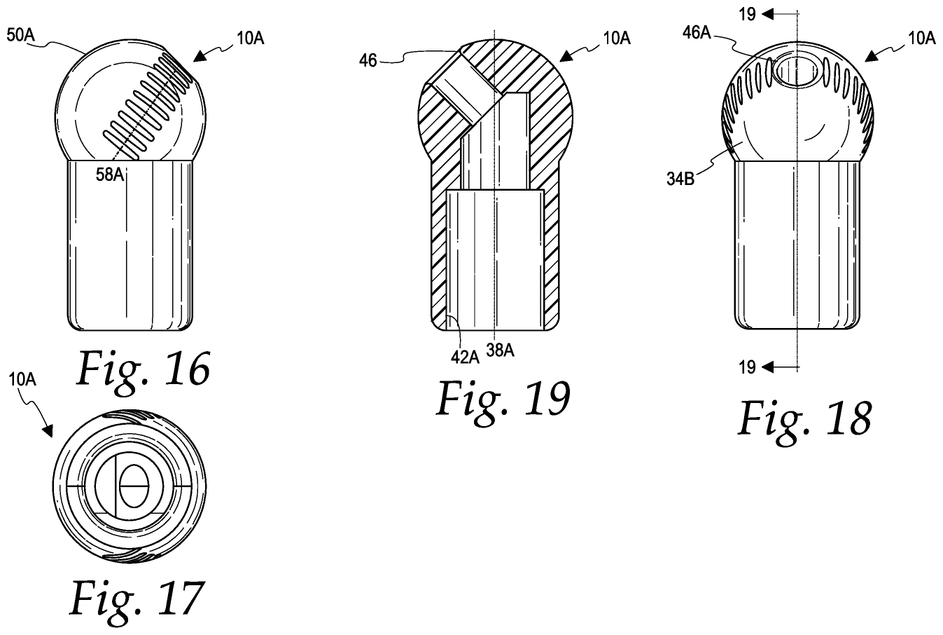

A second embodiment of a tip according to the present invention is shown in FIGS. 11-19, designated by the numeral 10A, and functions similarly to the first illustrated embodiment of the tip 10 as previously described in FIGS. 1-10. The numbered features of the second embodiment of the tip 10A illustrated in FIGS. 11-19 are analogous to features of the first embodiment of the tip 10 that share the same number, but without the suffix “A”. The second embodiment of the surgical tip 10A differs from the aforementioned first illustrated embodiment in that the second embodiment includes a plurality of recesses 54A in the terminal portion 34A, each having the form of a line or groove centered on an arc 58A (FIG. 16) on the spherical outer surface 50A that extends through the center of the port 46A. In one preferred form of the invention, the port 46A has a nominal diameter of about 0.30 mm and the recesses 54A each have a length of about 0.5 mm and a depth of about 0.04 mm.

The tip 10A is also hollow for being attached around the distal end of a cannula. Importantly, the tip 10A is formed from a polymer, preferably an elastomer such as silicone, which may be substantially softer and less rigid than the material of the cannula (which may be metal or a rigid or hard polymer) to permit the tip 10A to safely contact the lens capsule during use.

A desirable feature of the tip 10A is that the recesses 54A serve to better indicate to a user the location of the port 46A within the terminal portion 34A, which may be obscured or not visible during use. Furthermore, the elongate recesses 54A in the terminal portion 34A function to better prevent or at least minimize the adhesion of the emulsified nucleus during use of the tip 10A. In some forms, the recesses 54A may instead have the form of pigmentation, dye, or other marking on the surface 50A of the tip 10A.

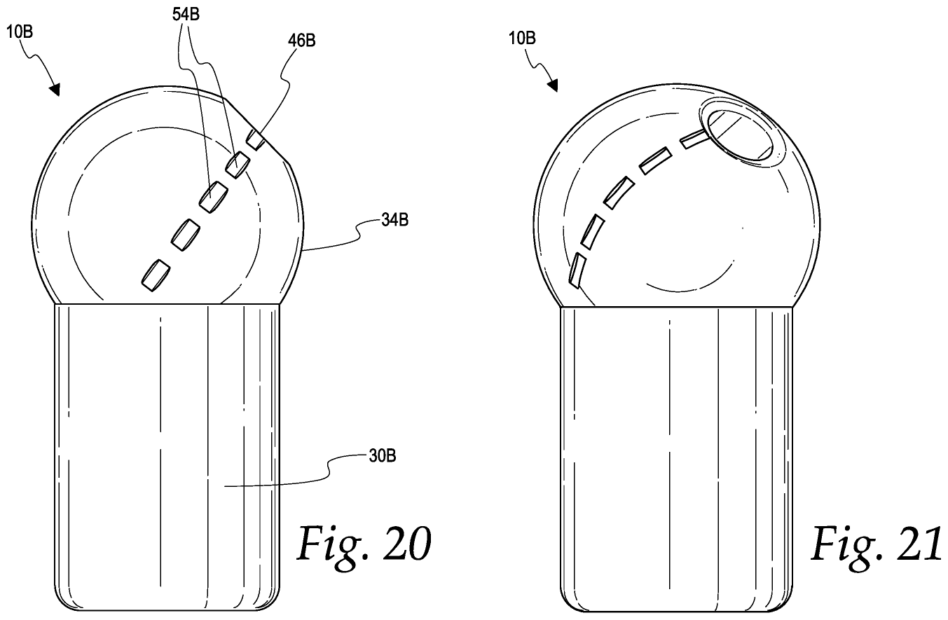

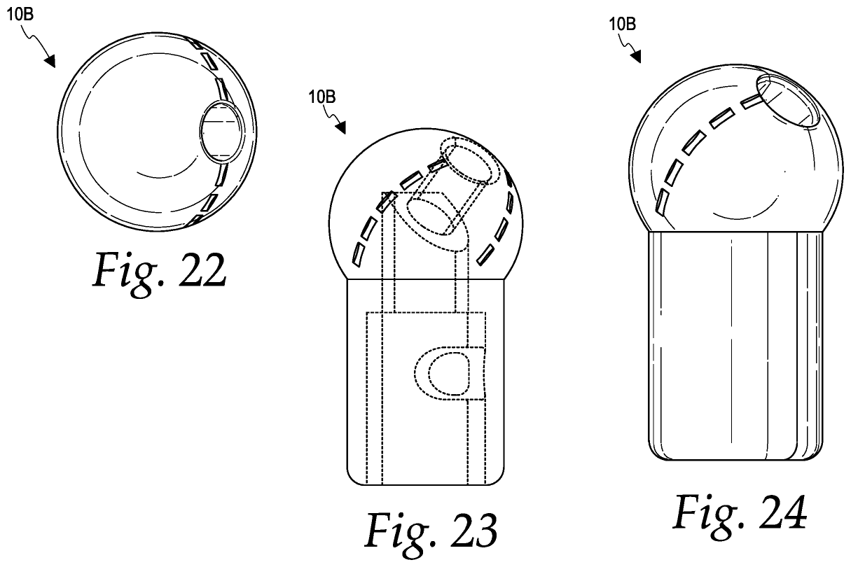

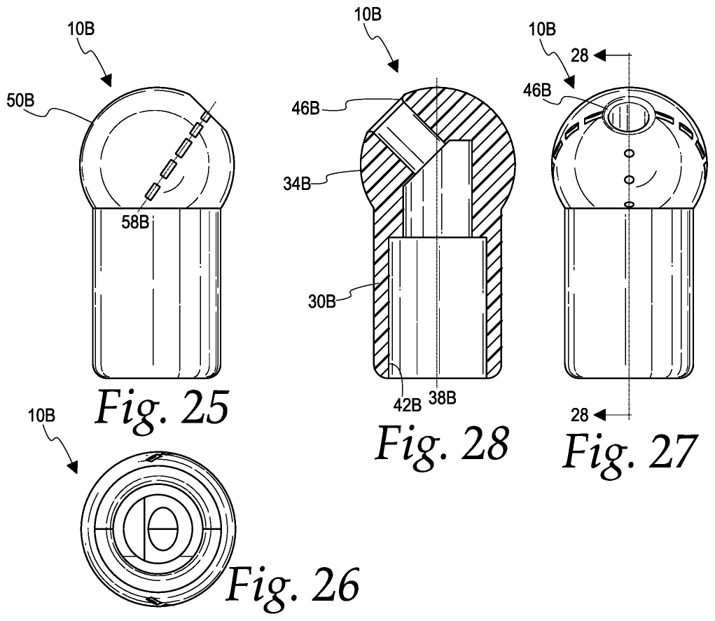

A third embodiment of a tip according to the present invention is shown in FIGS. 20-28, designated by the numeral 10B, and functions similarly to the first illustrated embodiment of the tip 10 as previously described in FIGS. 1-10. The numbered features of the third embodiment of the tip 10B illustrated in FIGS. 20-28 are analogous to features of the first embodiment of the tip 10 that share the same number, but without the suffix “B”. The third embodiment of the surgical tip 10B differs from the aforementioned first illustrated embodiment in that the third embodiment includes plurality of recesses 54B in the terminal portion 34B, each having the form of a wide and short line or groove centered on an arc 58B (FIG. 25) on the spherical outer surface 50B that extends through the center of the port 46B. In one preferred form of the invention, the port 46B has a nominal diameter of about 0.30 mm and the recesses 54B each have a length of about 0.05 mm and a depth of about 0.01 mm.

The tip 10B is also hollow for being attached around the distal end of a cannula. Importantly, the tip 10B is formed from a polymer, preferably an elastomer such as silicone, which may be substantially softer and less rigid than the material of the cannula (which may be metal or a rigid or hard polymer) to permit the tip 10B to safely contact the lens capsule during use.

A desirable feature of the tip 10B is that the recesses 54B serve to better indicate to a user the location of the port 46BA within the terminal portion 34B, which may be obscured or not visible during use. Furthermore, the elongate recesses 54B in the terminal portion 34B function to better prevent or at least minimize the adhesion of the emulsified nucleus during use of the tip 10B. In some forms, the recesses 54B may instead have the form of pigmentation, dye, or other marking on the surface 50B of the tip 10B.

A fourth embodiment of a tip according to the present invention is shown in FIG. 29 only, designated by the numeral 10C, and functions similarly to the first illustrated embodiment of the tip 10 as previously described in FIGS. 1-10. The numbered features of the fourth embodiment of the tip 10C illustrated in FIG. 29 are analogous to features of the first embodiment of the tip 10 that share the same number, but without the suffix “C”. The fourth embodiment of the surgical tip 10C differs from the aforementioned first illustrated embodiment in that the fourth embodiment includes plurality of recesses 54C covering a substantial portion (most) of the terminal portion 34C, each having the form of a circular dimple on the spherical outer surface 50C thereof.

The tip 10C is also hollow for being attached around the distal end of a cannula. Importantly, the tip 10C is formed from a polymer, preferably an elastomer such as silicone, which may be substantially softer and less rigid than the material of the cannula (which may be metal or a rigid or hard polymer) to permit the tip 10C to safely contact the lens capsule during use.

A desirable feature of the tip 10C is that the numerous recesses 54C serve to better prevent or at least minimize the adhesion of the emulsified nucleus during use of the tip 10C.

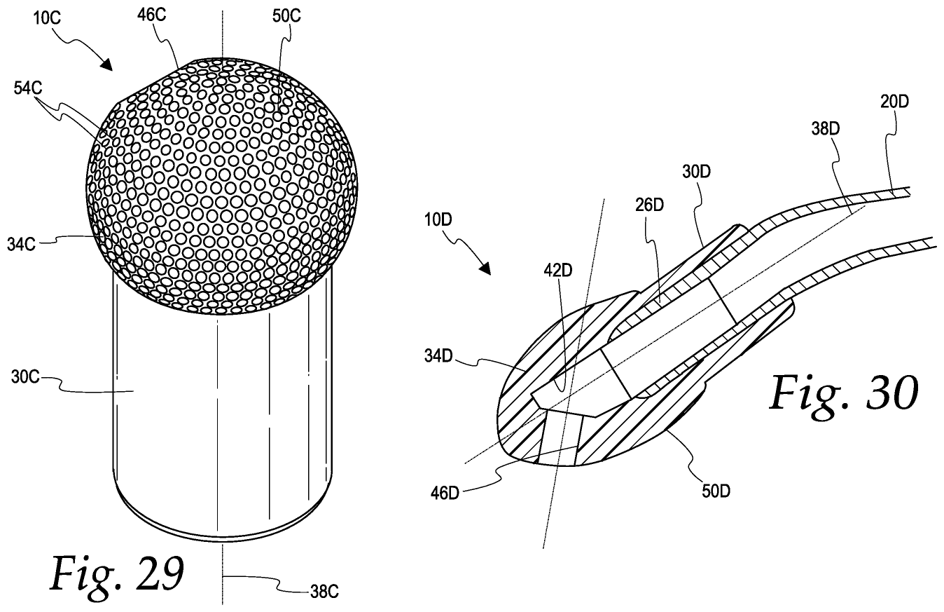

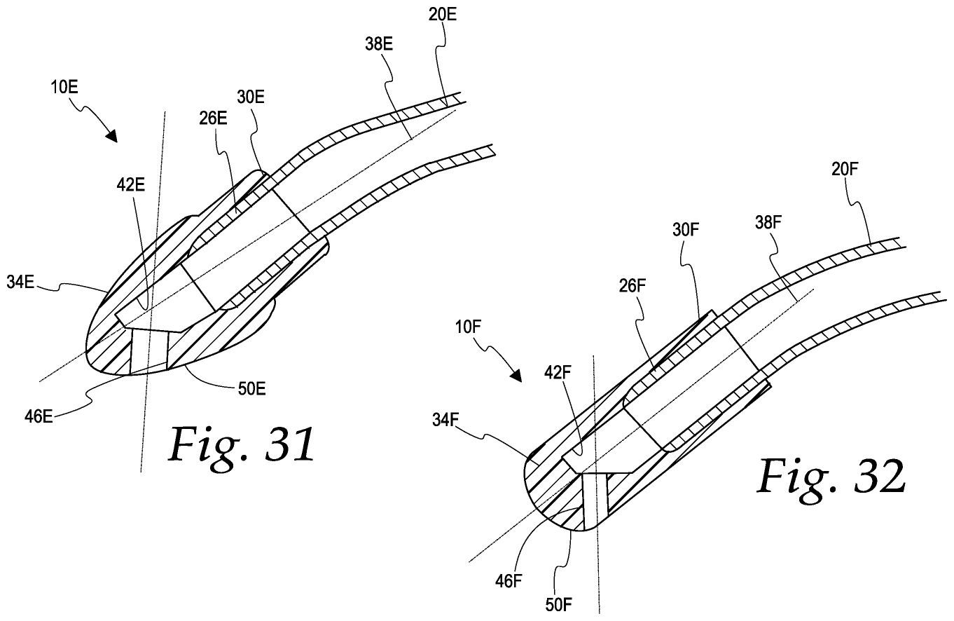

Fifth through eighth embodiments of tips according to the present invention are shown in FIGS. 30-33, designated by the numerals 10D, 10E, 10F, and 10G, respectively, which function similarly to the first illustrated embodiment of the tip 10 as previously described in FIGS. 1-10. The numbered features of the fourth embodiment of the tips 10D-10G illustrated in FIGS. 30-33 are analogous to features of the first embodiment of the tip 10 that share the same number, but without the suffixes “D-G”. The fifth through eighth embodiments of the surgical tip 10D-10G differ from the aforementioned first illustrated embodiment in the shape of the terminal portion 34D-34G. The terminal portion 34D-34G may have any combination of the above-discussed recess or recesses of the prior embodiments. The tips 10D-10G may be formed from a polymer, preferably an elastomer such as silicone, which may be substantially softer and less rigid than the material of the cannula (which may be metal or a rigid or hard polymer) to permit the tips to safely contact the lens capsule during use.

Referring to FIG. 30, the tip 10D, which is shown in cross-section, has a terminal portion 34D with an ovoid shape instead of a spherical shape. The port 46D is angled about 45 degrees relative to the central axis 38D of the sheath portion 30D. The cannula 20D is also preferably bent near the distal end 26D, but may be straight.

Referring to FIG. 31, the tip 10E, which is shown in cross-section, has a terminal portion 34E with a semi, or modified, -ovoid shape instead of a spherical shape. The port 46E is angled about 45 degrees relative to the central axis 38E of the sheath portion 30E. The cannula 20E is also preferably bent near the distal end 26E but may be straight.

Referring to FIG. 32, the tip 10F, which is shown in cross-section, has a terminal portion 34F with a blunted, rounded rectangular shape instead of a spherical shape. The port 46F is angled about 45 degrees relative to the central axis 38F of the sheath portion 30F. The cannula 20F is also preferably bent near the distal end 26F but may be straight.

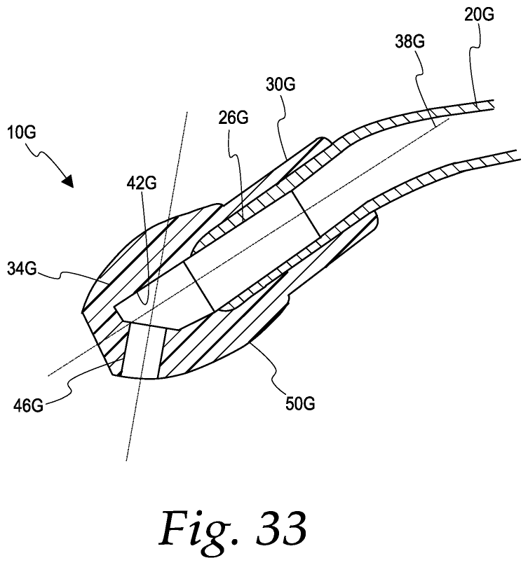

Referring to FIG. 33, the tip 10G, which is shown in cross-section, has a terminal portion 34G with a blunted, truncated ovoid shape instead of a spherical shape. The port 46G is angled about 45 degrees relative to the central axis 38G of the sheath portion 30G. The cannula 20G is also preferably bent near the distal end 26G but may be straight.

From the foregoing, it will be observed that numerous modifications and variations can be effected without departing from the true spirit and scope of the novel concept of the present invention. It is to be understood that no limitation of the broadest concepts with respect to the specific embodiments illustrated herein is intended or should be inferred. The disclosure is intended to cover, by the appended claims, all such modifications as fall within the scope of the claims.

1. A hollow tip for use with a cannula, said tip comprising:
a sheath portion for being affixed around the cannula, said sheath portion defining a central axis; and
a terminal portion extending from said sheath portion along said central axis, said terminal portion and said sheath portion defining an internal passage communicating with at least one port in said terminal portion to permit fluid communication through said tip, said terminal portion including at least one recess therein for indicating the location of said at least one port and/or inhibiting adherence of eye tissue to said tip.

2. The tip in accordance with claim 1 in combination with a cannula and a handpiece connected to a vacuum source and/or an infusion fluid supply source.

3. The tip in accordance with claim 1, wherein said at least one recess has the form of a plurality of recesses.

4. The tip in accordance with claim 3, wherein said plurality of recesses extend along an arc extending through a center of said at least one port.

5. The tip in accordance with claim 3, wherein said plurality of recesses are circular.

6. The tip in accordance with claim 3, wherein said plurality of recesses are elongate.

7. The tip in accordance with claim 3, wherein said plurality of recesses cover substantially all of said terminal portion.

8. The tip in accordance with claim 1, wherein said sheath portion defines a first diameter and said terminal portion defines a second diameter that is greater than said first diameter.

9. The tip in accordance with claim 1, wherein said at least one port is angled between about 40 degrees and about 50 degrees relative to said central axis.

10. The tip in accordance with claim 1, wherein said tip is formed from an elastomer.

11. The tip in accordance with claim 1, wherein said terminal portion defines an outer surface that is relatively rougher than said sheath portion.

12. The tip in accordance with claim 1, wherein said terminal portion is spherical.

13. The tip in accordance with claim 1, wherein said terminal portion is ovoid.

14. The tip in accordance with claim 1, wherein said terminal portion is semi-ovoid.

15. The tip in accordance with claim 1, wherein said terminal portion is a rounded rectangular parallelepiped.

16. The tip in accordance with claim 1, wherein said terminal portion is truncated at a distal end thereof.

17.-32. (canceled)
